(19) United States
(12) Patent Application Publication
(10) Pub. No.: US 2009/0053103 A1
(43) Pub. Date: Feb. 26, 2009

(54) Non-linting sterilization packaging material

(76) Inventors: William Patrick Mortimer, Northeast, MD (US); Alex R. Hobson, Elkton, MD (US); Christopher H. Brennan, New Castle, DE (US)

Correspondence Address: GORE ENTERPRISE HOLDINGS, INC., 551 PAPER MILL ROAD, P. O. BOX 9206, NEWARK, DE, 19714-9206, US

(21) Appl. No.: 11/894,238

(22) Filed: Aug. 20, 2007

(51) Int. Cl. A61L 2/20; B32B 3/24; B32B 5/22

(52) U.S. Cl. 422/28; 206/439; 428/138; 442/76

(57) ABSTRACT

Sterilization packaging materials of a microporous membrane and support layer which are bacteria impermeable, gas permeable, non-linting and compatible with all gas sterilization systems are provided. Desirable translucent areas may be incorporated into the sterilization packaging materials. Sterilization packages of these materials and methods for their use in gas sterilization of articles are also provided.

### FIELD OF THE INVENTION

The present invention relates to a sterilization packaging material comprising a porous PTFE membrane and a porous PTFE membrane laminate. The packaging material may incorporate translucent portions, and is useful in multiple gas sterilization processes.

### BACKGROUND OF THE INVENTION

Sterilization of instruments, devices and materials used in surgery is critical to the safety and well being of the patient. Ensuring that the contents within a sterilization package are completely sterile is an ever growing concern. There are a variety of different sterilization methods each with their own set of benefits and challenges. In gas sterilization processes, a sterilizing gas such as hydrogen peroxide, ethylene oxide, steam or ozone, is used to destroy the bacteria that may exist on the item or items to be sterilized. The items to be sterilized are placed in an enclosure such as a tray, pouch or container that contains or is sealed with a gas permeable sterilization packaging material. Gas permeable sterilization packaging materials allow the sterilization gas to pass through and act as a barrier to bacteria after the enclosure is removed from the sterilizer. In the case of a pouch, one side of the pouch is typically gas permeable and the other side is a non-permeable film such as Polyester (PET) or polyethylene naphthalate (PEN). In the case of a container, a gas permeable sheet of material is typically sealed to or within the lid. In the case of the tray, a gas permeable sheet of material is wrapped around the tray.

Sterilization packaging materials should first and foremost be a barrier to microbes in order to prevent any contamination of the contents of the sterilization package after removal from the sterilization unit. In addition, the sterilization packaging material should be clean, or non-linting to prevent the physical contamination of the contents within the sterilization package.

Further, for the sterilization cycle to be efficacious, the sterilization packaging material must have adequate gas permeation to allow the sterilization gas to reach the contents inside the enclosure and must not be too reactive with the sterilization gas, thereby reducing the concentration and possibly compromising the efficacy of the sterilization cycle. However, all gas sterilization methods expose the sterilization packaging material to either aggressive oxidation gases or high temperatures. Ozone based sterilization, a relatively new method with important benefits with respect to cycle time and environment impact, is particularly aggressive with respect to sterilization packaging materials. In ozone based sterilization systems, such as the 125L Ozone Sterilizer, available from TSO3, Quebec, Canada, ozone gas and water vapor are introduced into the sterilization chamber at low temperature and the combination of the two gases quickly and efficaciously sterilizes the contents within the sterilization package. However, because of the combination of both ozone and water vapor, the sterilization packaging material must not only be non-reactive with ozone, but also sufficiently hydrophobic. The sterilization packaging material is wrapped around contents to be sterilized thus forming a sterilization enclosure.

It is critically important to be certain that the contents in the sterilization enclosure are effectively sterilized since in many cases the contents will be used in medical surgery. In most sterilization processes, an indicator is placed inside the sterilization enclosure along with the materials to be sterilized. This chemical indicator changes color when exposed to the sterilization gases, indicating an effective sterilization cycle. After the sterilization enclosure is removed from the sterilizer, it is typically placed on a shelf until the contents are needed. If upon opening the enclosure, the indicator has not changed color indicating an effective sterilization cycle, the enclosure must be removed from the operating room, and another enclosure must be brought in. This delay in surgery can be life threatening. It is therefore desirable to be able to view the change in color of the indicator through the sterilization enclosure without opening it. Some sterilization containers and pouches have transparent elements to allow viewing of the indicator. However, current sterilization wrap materials do not provide viewing of the indicator through the wrap material.

In addition, many of the instruments or devices that are routinely sterilized have identification codes such as numbers or bar codes. As with the indicators, it would be of value to be able to determine or verify the contents, including the identification codes, within a sterilization enclosure without opening or unwrapping it. It is therefore beneficial for sterilization packaging materials to provide for at least a portion of the enclosure to be seen through.

Finally, in the case of a sterilization wrap material, a sheet of material has to be stiff enough so that is does not drape into the open top of the tray and cause open areas between the layers of the folded wrap material. On the other hand, the sterilization wrap material has to be supple enough such that it will lie flat after the tray is unwrapped in the operating room. The inside surface of the sterilization wrap is commonly used as a sterile surface within the operating room where instruments can be temporarily placed. It is desirable that the sterilization wrap material have very little fold setting, remaining in the wrap upon or after opening.

There are few materials that possess the required unique combination of chemical inertness, gas permeability, stiffness, non-linting and microbial resistance to be useful as sterilization packaging materials.

A three-layer laminate of expanded polytetrafluoroethylene (ePTFE) located between a non-woven sheet and a grid patterned adhesive layer which provides adequate permeation to sterilization gases and resistance to penetration by bacteria has been disclosed in U.S. Pat. No. 5,342,673 to Bowman et al. However, the packaging material described in U.S. Pat. No. 5,342,673 does not provide a non-linting inner surface and would not be compatible with the broad range of sterilization methods disclosed herein because of the adhesive layer. Furthermore, the sterilization material described in U.S. Pat. No. 5,342,673 does not provide any translucent areas for viewing the contents of the sterilization enclosure.

Additionally, most commercially available sterilization packaging materials to date can not be used in all of the different gas sterilization processes described. Some of the commercially available materials work well in one or two of the gas sterilization processes but degrade or are not thermally stable enough for the others. For example, melt-blown polypropylene sterilization packaging material is chemically compatible with both hydrogen peroxide and ethylene oxide sterilization methods, but degrades in the ozone sterilization process and can be thermally degraded if steam sterilization temperatures are not carefully controlled. Likewise, spunbond olefin such as TYVEK® available from Dupont Inc., Wilmington, Del., can withstand the temperatures of the steam sterilization process and is chemically compatible with both hydrogen peroxide and ethylene oxide but is too hydroscopic for the ozone sterilization method. Hydroscopic sterilization packaging materials such as those made from nylon, or paper, are also not suitable as sterilization wraps for ozone sterilization because they significantly limit the number of packages that can be sterilized at one time.

Many hospitals have multiple types of gas sterilization processes and because of the compatibility deficiencies in the flexible sterilization packaging materials used to wrap instruments, they have to stock specific materials for each sterilization process. This is expensive and complicated for procurement and for the clinicians responsible for proper sterilization processing.

Higher Log Reduction Values (LRVs) are preferred to minimize microbe passage through the sterilization packaging material. The currently available flexible sterilization packaging materials provide LRV values ranging from 2 to 5.5, but do not provide LRVs greater than 5.5 (as determined with the Microbial Barrier test ASTM-F1608). Higher LRV values indicate improvement of the barrier properties to microbes. A flexible barrier to microbes, that is permeable to sterilant with an LRV equal to or greater than 2.5, is achieved by the present laminate. It is further possible to achieve an LRV equal to or greater than 6.0 with the present laminate. Some of the non-wovens or paper materials used as sterilization packaging materials are made up of small discrete fibers that are bonded together to make a continuous sheet. These discrete fibers can become dislodged from the material and lint onto the articles with the sterilization package. In general, as the weight of the non-wovens or papers increase, so do the microbial barrier properties. However, as the weight increases so does the stiffness of the material, which is not desirable. In addition, many of the sterilization packaging materials become physically degraded after being exposed to the sterilization gases which cause them to have an increased propensity to lint. There exists a need for a flexible sterilization packaging material with an LRV greater than 2.5 which is non-linting before and after the sterilization process.

In addition, sterilization pouches for trays are available with a clear plastic side and a permeable side for viewing of the sterilization indicator and/or instrument codes. However, in the case of trays wrapped with a sterilization packaging material, there is typically no way of knowing the contents within the container or if the contents have been effectively sterilized without opening the package.

As described in the article Choosing a Sterilization Wrap for Surgical Packs by William A. Rutala, PhD, MPH, and David J. Weber, MD, MPH, the ideal sterilization wrap material would have all of the following combinations of properties. There are currently no sterilization wrap materials, but for the present invention, that provide this unique combination of properties.

TABLE 1
Characteristics of an Ideal Sterilization Wrap

| Characteristic | Goal |
|---|---|
| Barrier effectiveness | Ability to prevent microbial penetration |
| | and maintain sterility of surgical pack and prevents |
| | penetration of liquids (i.e., repellent) |
| Penetrability (steam) | Allows steam to penetrate |
| Penetrability (e.g., | Allows sterilizing gases or plasmas to penetrate |
| ethylene oxide) | |
| Aeration | Allows aeration post-sterilization (e.g., allows |
| | ethylene oxide to dissipate) |
| Ease of use | Easy to use by personnel |
| Drapeability | Conforms to equipment pack contours smoothly |
| | and closely |
| Flexibility | Enough sizes to accommodate any sized or |
| | shaped item |
| Puncture resistance | Resists punctures |
| Tear strength | Resists tears |
| Toxicity | Non-toxic |
| Odor | No odor |
| Waste disposal | Adheres to local and state solid waste disposal rules |
| Linting | Minimal linting during use |
| Cost | Low cost in use |
| Transparency | Allows verification of pack's internal contents |
| | before opening the pack |

Accordingly, there exists a need for a flexible sterilization packaging material that is a barrier to microbes having an LRV of greater than 2.5, and which can be used in all gas sterilization systems, is non-linting, is translucent to allow for the identification of codes and color changes of indicators with the sterilization package, and is stiff enough to function as a sterilization wrap but supple enough not to take on a fold set. The sterilization packaging material which is the subject of this application fulfills this need.

### SUMMARY OF THE INVENTION

The present invention relates to sterilization packaging materials comprising expanded polytetrafluoroethylene, herein referred to as “ePTFE”, membranes or porous ePTFE membranes affixed to support layers which provide a barrier to microbes having an LRV of greater than 2.5, are non-linting, can be used in all gas based sterilization systems, and are translucent at least in part to allow for the identification of instrument identification codes and color changes of effective sterilization indicators within the sterilization package.

The present invention also relates to sterilization packages comprising these sterilization packaging materials and method for use of these sterilization packaging materials and packages in gas permeable sterilization of instruments and devices.

### BRIEF DESCRIPTION OF THE FIGURES

FIG. 1 is a drawing representing a cross-sectional view of a sterilization packaging material with a microporous membrane affixed to a support layer.

FIG. 2 is a drawing representing a cross-sectional view of a sterilization packaging material with a microporous membrane affixed to both sides of a support layer.

FIG. 3 is a drawing representing a cross-sectional view of a lamination process for affixing the microporous membrane to a support layer as depicted in FIG. 1.

FIG. 4 is a drawing representing a cross-sectional view of a compression process for making the sterilization packaging material of the present invention.

FIG. 5a is a drawing representing a cross-sectional view of a sterilization package with a sterilization packaging material sealing the sterilization enclosure. Inside the sterilization enclosure is an instrument with an identification code and an effective sterilization indicator.

FIG. 5b is a drawing representing a top view of the sterilization package depicted in FIG. 5a with a sterilization packaging material having oval translucent areas for visualizing an instrument identification code and an effective sterilization indicator.

FIG. 6 is a drawing of an exemplary pouch enclosure comprising the sterilization packaging material of the present invention.

FIG. 7 is a drawing of an exemplary container enclosure comprising the sterilization packaging material of the present invention.

FIG. 8 is a drawing of an exemplary tray enclosure comprising the sterilization packaging material of the present invention.

FIG. 9 is a drawing representing a cross-sectional view of a compression and heating process for making the sterilization packaging material of the present invention.

### DEFINITIONS

As used in this application the term “mass area” refers to the mass per unit area of a material and is expressed in grams per square meter.

As used in this application, the term “gas sterilization system” refers to any sterilization system using a gas as the sterilant. Examples include, but are not limited to, hydrogen peroxide, ethylene oxide, steam and ozone sterilization systems.

As used in this application, the term “microporous membrane” refers to any porous substrate that has a mean pore flow size of less than 1 um, as described by the Bubble Point Procedure described in the test methods section herein.

As used in this application, the term “bacteria impermeable” material refers to any gas permeable material that has an LRV value of greater than 6 as determined by ASTM F1608 Microbial Ranking Test. ePTFE membranes alone and ePTFE membrane affixed to support layers are non-limiting examples of bacteria impermeable material as defined herein.

As used in this application, the term “enclosure” refers to a tray, pouch, or any other type of container that is used to hold items to be sterilized.

As used in this application, the term “partially imbibed” means reducing the porosity of a microporous membrane through the filling of at least a portion of the pores in the membrane with a polymer. The polymer may be imbibed into the microporous membrane through various processes including, but not limited to, solution coating, melt imbibing and combinations thereof.

As used in this application, the term “translucent” means that the material has a light transmission value of at least 30% as measured with the light transmission method described herein.

As used in this application, the term “non-linting” means that less than 500 particles per minute are released from the material as measured using the Particle Shed Analysis Test with the Helmke-Drum test protocol under test code PSA-110 at Nelson Labs, Salt Lake City, Utah.

As used in this application “flexible sterilization packaging” is a material which may be conformed to accommodate different sizes or shapes of instruments, by folding and securing the wrap around the instruments. A typical wrap is a sheet or roll good material. Flexibility of less than 1000 grams as determined by ASTM D6828-02 is desired.

### DETAILED DESCRIPTION OF THE INVENTION

The sterilization packaging materials of the present invention provide a unique combination of properties that make them ideal for use in all gas sterilization systems, especially for ozone sterilization where other commercially available materials do not have the properties necessary for them to be used as sterilization wrap.

The flexible sterilization packaging material of the present invention comprises a microporous membrane as a bacterial impermeable layer which can be affixed to a support layer. The microporous membrane is typically an expanded PTFE (ePTFE) membrane. Microporous ePTFE can be made to have a structure that is tight enough to prevent bacteria from passing through. Specifically, microporous ePTFE can be prepared having an LRV of 3.0 or greater, as measured by ASTM F1608, while also having adequate air permeability to allow the sterilizing gas to effectively flow through. In addition, ePTFE membrane is chemically inert, and thermally stable, so it can withstand the harsh environments of all of the gas sterilization processes including but not limited to hydrogen peroxide, ethylene oxide, steam and ozone. Because ePTFE membrane is chemically inert, and because it is an expanded polymer material with an interconnected network of fibrils, it is an ideal non-linting material for sterilization applications as well. Exemplary methods for production of ePTFE can be found in the art and particularly as described in U.S. Pat. No. 3,953,566, and U.S. Pat. No. 5,476,589. Furthermore, the ePTFE membrane or a portion of the ePTFE membrane can be made to be translucent in desired areas, through imbibing or compression, or as described in U.S. Patent Publication 2007/0012624A1 to Bacino.

An ePTFE membrane is typically thin and very supple. Thus, when used alone as a sterilization wrap material, it drapes into the open top of the enclosure. In addition, the ePTFE membrane alone is prone to tearing or ripping when used as a sterilization wrap and is therefore not traditionally preferred as a sterilization wrap material. It has been found that even though ePTFE was thought to be too thin to be used as an inner wrap layer for sterilization packaging, if the ePTFE is affixed to a support layer, it becomes an ideal wrap. However, the support backing layer does not provide non-linting or microbial barrier properties. Support layers useful in the present invention must be comprised of a material capable of providing physical support to the ePTFE membrane and increasing the stiffness of the sterilization packaging material while being compatible with gas sterilization systems. Support layers for use in the present invention can comprise a wide range of materials including, but not limited to, ePTFE membranes or tapes, non-wovens, spunbonded, melt-blown, hydroentangled, open cell foams, woven fabric, paper, netting and the like, and plastics including but not limited to polypropylene, polyester, nylon, olefins, FEP, PFA, PVDF, and CTFE, and combinations thereof. A preferred material for the support layer used in the present invention is a non-woven material comprising a bi-component fiber with a polyethylene sheath over a polyester core, such as S222 from HDK Industries, Rogersville, Tenn. Other chemically inert materials preferred as support layers include, but are not limited to, ePTFE membrane and FEP netting material.

An ideal wrap material has enough stiffness to prevent draping or pouching over the open area of the tray, and is supple enough not to retain a fold set when the package is unwrapped. If the material is too supple, it will drape over the opening of the tray which may provide a pathway for bacteria to penetrate the interior of the wrapped tray. When the sterilization package is unwrapped in the operating room, it is important that the sterilization wrap lays flat and does not retain a fold set causing the material to protrude up. It is common to use the interior surface of the sterilization wrap material after unwrapping in the operating room, as a sterilized surface on which to temporarily place instruments or tools. Therefore, a retained fold set in the sterilization wrap material is not preferred, as it prevents the wrap from flattening.

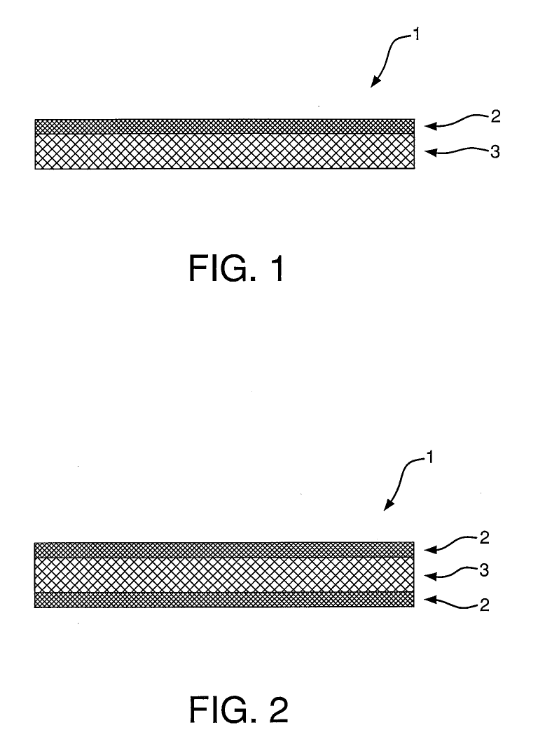

As shown in FIG. 1, a first microporous membrane 2 can be affixed to just one side of the support layer 3, as depicted in FIG. 1. It has not been taught to use only one support layer on a microporous membrane such as ePTFE membrane so that a sterilization packaging material may optimize the set folding and drape prevention aspects required. Alternatively, first and second microporous membranes 2 can be affixed to both sides of the support layer 3 as depicted in FIG. 2.

The microporous membrane can be affixed to the support layer through any number of standard processes including, but not limited to, hot roll lamination, ultrasonic bonding or laminating, point bonding or laminating, adhesive bonding, and the like. The microporous membrane can be affixed to the support layer across the entire surface of the sterilization packaging material or along the edges of the sterilization packaging material, or in continuous or discontinuous pattern.

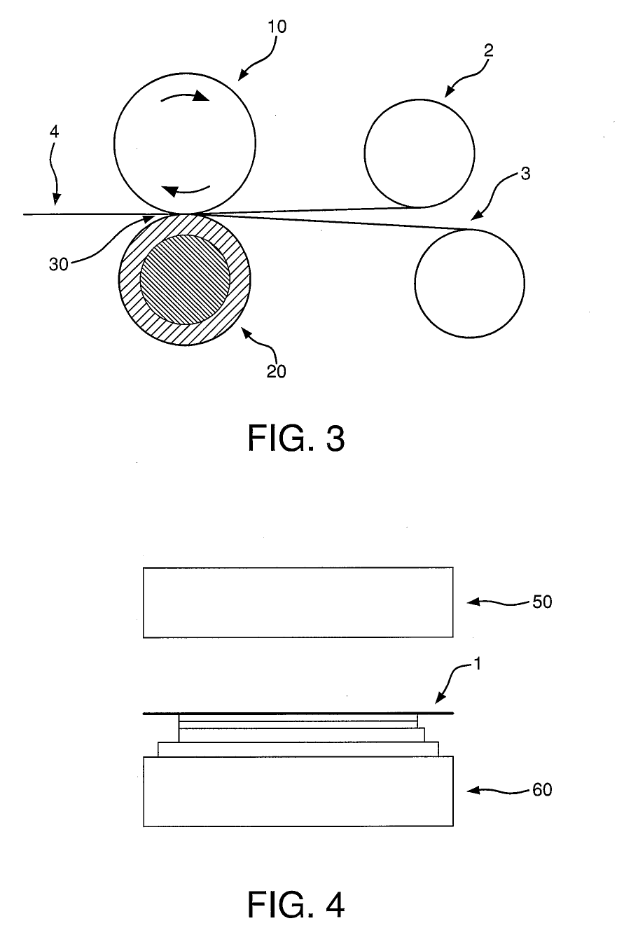

A preferred method of affixing the microporous membrane to the support layer is through hot roll lamination as depicted in FIG. 3. In hot roll lamination, the microporous membrane 2, is layered with support layer 3 and fed through a heated roll 10 and an elastomer roll 20. The elastomer roll 20 is pressed against the hot roll 10 to provide sufficient pressure for bonding the layers of material as they pass through the nip 30. The hot roll lamination process creates a laminate 4 of microporous membrane 2 affixed to a support layer 3.

Microporous membranes can be affixed to both sides of the support layer through any suitable method or combination of methods as described above, as well. A preferred method of affixing microporous membranes to both sides of the support layer is by running the laminate 4 as depicted in FIG. 3 through the hot roll lamination process a second time and affixing a second microporous membrane to the other side of the support layer.

The sterilization packaging material of the present invention can be used as a gas permeable, bacteria impermeable sterilization wrap for any sterilization enclosure including, but not limited to pouches, containers, and trays.

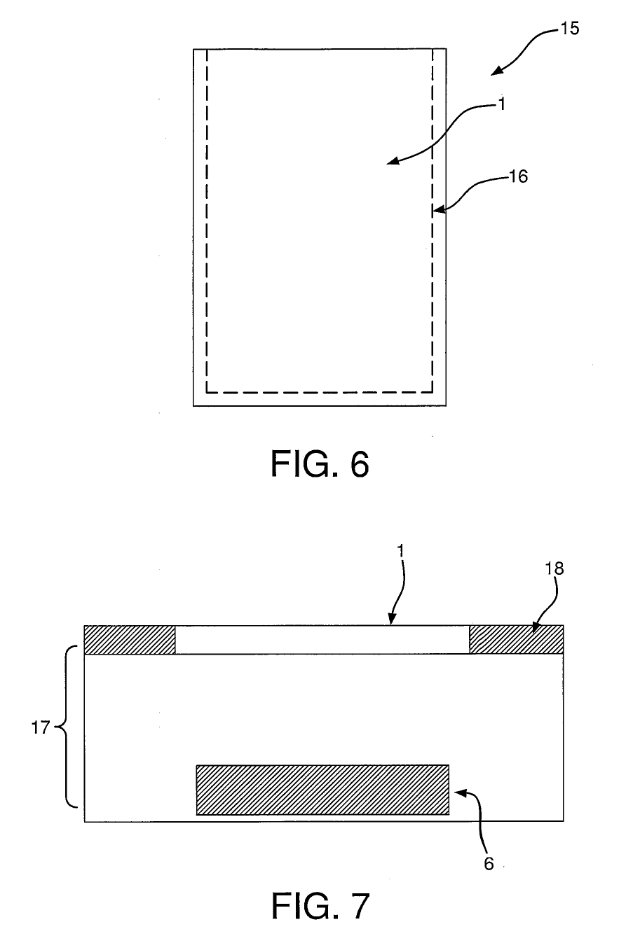

When the sterilization packaging material is used as the gas permeable layer in a sterilization enclosure such as a pouch as depicted in FIG. 6, the sterilization packaging material 1 is bonded to form a seal 16 with a second layer around the periphery to form a pouch 15. When the sterilization packaging material is used in this pouch configuration, it can be stiffer than when it is used as a sterilization wrap material. The second layer in the pouch configuration can include another layer of sterilization wrap material of the present invention, or it can be selected from a wide range of materials including non-wovens, wovens, or non-permeable films. Typically, the second layer of the pouch is a transparent non-permeable film layer. Some of the preferred film materials for use as the second layer in the pouch configuration include, but are not limited to, polytetrafluoroethylene (PTFE), fluorinated ethylene propylene or FEP, perfluoroalkoxy copolymer or PFA, PEN, polyethylene terephthalate, polypropylenes, polyethylenes, polyamides, and mylar. Chemically inert film layers are cost prohibitive and lower cost materials such as PET or PEN are used; however, these materials cannot be used as a flexible wrap material because of low permeation to sterilant gases.

When the sterilization packaging material is used as the gas permeable layer in a sterilization enclosure such as a container as depicted in FIG. 7, the sterilization packaging material 1 is typically placed and sealed in or onto the lid 18 of the container 17 with an article 6 inside the container 17 prior to sealing the lid 18. Since the sterilization packaging material used in a container does not need to be supple, a wider array of support layers are available. In this embodiment, support materials that provide better sealing properties may be preferred. In addition, the support material in this configuration may only extend around the periphery of the sterilization packaging material or in the area of the seal.

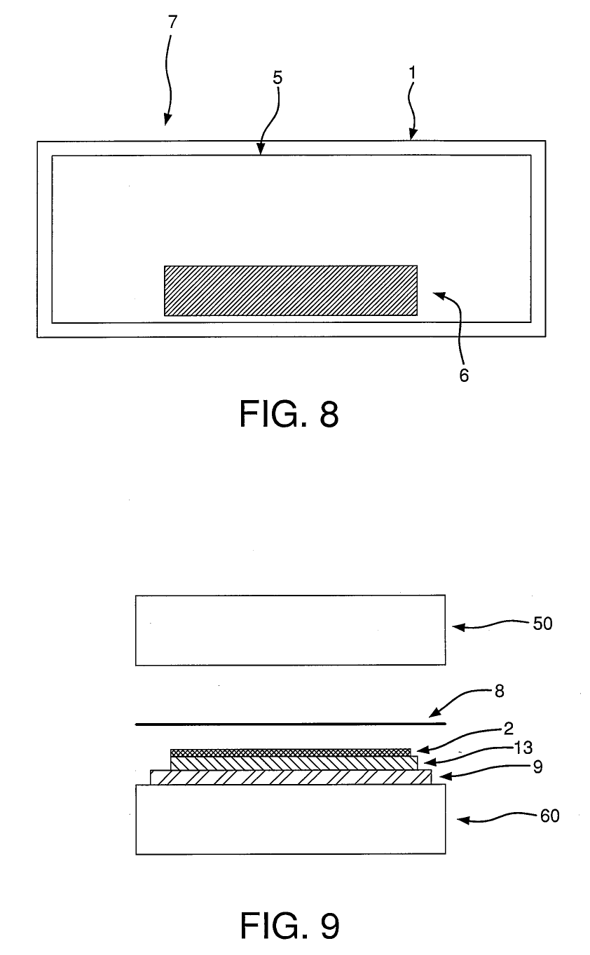

When the sterilization packaging material of the present invention is used as the gas permeable layer in a sterilization enclosure such as a tray, as depicted in FIG. 8, the sterilization packaging material 1 is wrapped around the tray 5 containing an article 6 to be sterilized so that the tray 5 is completely covered thereby forming a sterilization package 7. In this embodiment, the sterilization wrap material includes a microporous membrane and a support layer, such that it is stiff enough not to drape into the open top of the tray and supple enough to lay flat when the package is opened. In addition, when wrapping a tray with the sterilization packaging material, the microporous membrane side should be oriented toward the inside of the tray, and the support material should be oriented toward the outside of the tray. The microporous membrane provides a non-linting surface and protects the article inside the tray from being contaminated with lint from the support layer.

In one embodiment, the sterilization packaging material of the present invention is made to be translucent by bonding a translucent ePTFE membrane to a translucent support layer such as a netting or thin non-woven.

In another embodiment, the sterilization packaging material is made to be translucent in discrete areas through post processing. Regions of translucency can be obtained, for example, by a process wherein selected regions of the sterilization packaging material are partially or fully imbibed with a polymer. For example, microporous ePTFE is visually white because of the difference in the index of refraction between PTFE and air. This difference in index of refraction diffusely reflects light making the microporous ePTFE appear white. However when the porosity of the ePTFE membrane is reduced through compression, or when pores of the ePTFE are imbibed with another polymer, the ePTFE membrane becomes translucent.

In one embodiment, the process of partial imbibing comprises coating a region of the microporous membrane with a polymer dissolved in solvent and subsequently removing the solvent. The polymer of the coating at least partially fills the voids of the microporous membrane thereby rendering those coated regions more translucent. Alternatively or in addition, the imbibing polymer may be heated to a temperature above the melt temperature of the polymer and coated onto a region of the microporous membrane. In another embodiment, the imbibing polymer may be in the form of a discontinuous film and may be laminated to the microporous membrane and subsequently heated and melted into the microporous membrane to create regions of different translucency.

Alternatively, the support layer of the sterilization packaging material 1 may be heated and compressed with the microporous membrane as depicted in FIG. 4 between a top plate 50 and a bottom plate 60 so that the substrate layer melts into the microporous membrane and at least partially fills the void volume of the microporous membrane. To render regions of the sterilization packaging material more translucent, the support material may be patterned or may be discontinuous, or a patterned roller may be used to press the two layers together.

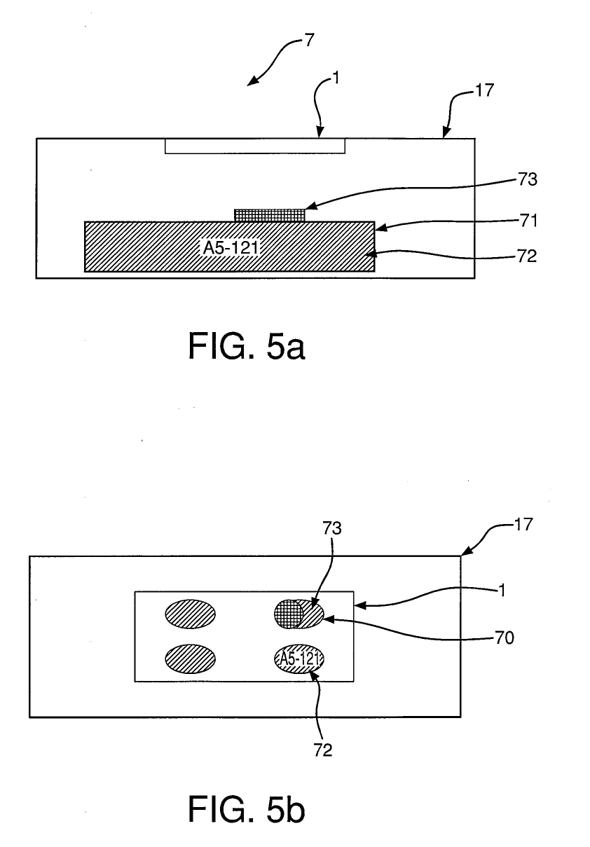

Exemplary sterilization packages with sterilization packaging material with translucent portions are depicted in FIGS. 5a and 5b. As shown in these figures, areas or translucency 70 in the sterilization packaging material 1 of the sterilization package 7 provide for visualizing an instrument identification code 72 of a medical instrument 71 and a sterilization enclosure indicator 73 within the sterilization enclosure; in this example a container 17.

The surface of either the support layer or the membrane may be modified to produce a less smooth surface, thereby increasing the ease of handling the packaging material. Wrap techniques known in the industry can be used to seal the sterilization packaging materials, such techniques include, but are not limited to, envelope wrap protocol procedures.

Thus, the present invention provides a sterilization packaging material for use in sterilization packages and in methods of sterilization involving gas sterilization systems. It further provides a highly desired non-linting aspect along with the ability to incorporate translucent areas.

The following non-limiting exemplary test methods were used to evaluate the sterilization packaging material and sterilization packages of the present invention.

### Test Methods

### Log Reduction Value (LRV)

Aerosol Spore Challenge Testing, ASTM F1608, was conducted by Nelson Labs according to protocol number 200700151 Revision 00. The values reported in this application are the average of four tests for each material type. In this test, samples of porous materials are subjected to an aerosol of Bacillus subtilis var. niger spores within an exposure chamber. Spores which pass through the porous sample are collected on membrane filters and enumerated. The logarithm reduction value (LRV) is calculated by comparing the logarithm of the number of spores passing through the porous material with the logarithm of the microbial challenge.

### Particle Shed Analysis

Particle shed analysis testing was conducted using the Helmke Drum Particle Test, Test Code PSA 110 at Nelson Labs, Salt Lake City, Utah. The test determines the particle counts on cleanroom attire, wipers, instrument wraps, and similar woven and non-woven fabrics. The procedure consists of placing the test material or garment into a stainless steel drum which rotates at 10 revolutions per minute (RPMs) and is connected to a laser particle counter.

### Tensile Break Load Measurements and Matrix Tensile Strength (MTS) Calculations

Tensile break load was measured using an INSTRON 1122 tensile test machine equipped with flat-faced grips and a 0.445 kN load cell. The gauge length was 5.08 cm and the cross-head speed was 50.8 cm/min. The sample dimensions were 2.54 cm by 15.24 cm. For longitudinal MTS measurements, the larger dimension of the sample was oriented in the machine, or “down web,” direction. For the transverse MTS measurements, the larger dimension of the sample was oriented perpendicular to the machine direction, also known as the cross-web direction. Each sample was weighed using a Mettler Toledo Scale Model AG204, then the thickness of the samples was taken using the Kafer FZ1000/30 thickness snap gauge. The samples were then tested individually on the tensile tester. Three different sections of each sample were measured. The average of the three tests in both the longitudinal and transverse directions was used to determine the maximum load and elongation at maximum load reported in Table 4.

### Gurley Measurements

The gurley air flow test measures the time in seconds for 100 cm³ of air to flow through a 6.45 cm² sample at 12.4 cm of water pressure. The samples were measured in a gurley densometer Model 4340 Automatic Densometer.

### Bubble Point Measurements

The bubble point and mean flow pore size were measured according to the general teachings of ASTM F31 6-03 using a Capillary Flow Porometer (Model CFP 1500 AEXL from Porous Materials Inc., Ithaca, N.Y.). The sample membrane was placed into the sample chamber and wet with SilWick Silicone Fluid (available from Porous Materials Inc.) having a surface tension of 19.1 dynes/cm. The bottom clamp of the sample chamber had a 2.54 cm diameter, 3.175 mm thick porous metal disc insert (Mott Metallurgical, Farmington, Conn., 40 micron porous metal disk) and the top clamp of the sample chamber had a 3.175 mm diameter hole. Using the Capwin software version 6.62.1 the following parameters were set as specified in Table 2. The values presented for bubble point and mean flow pore size were the average of two measurements.

TABLE 2

| Parameter | Set Point | Parameter | Set Point |
|---|---|---|---|
| maxflow (cc/m) | 200000 | mineqtime (sec) | 30 |
| bublflow (cc/m) | 100 | presslew (cts) | 10 |
| F/PT (old bubltime) | 40 | flowslew (cts) | 50 |
| minbppres (PSI) | 0 | eqiter | 3 |
| zerotime (sec) | 1 | aveiter | 20 |
| v2incr (cts) | 10 | maxpdif (PSI) | 0.1 |
| preginc (cts) | 1 | maxfdif (cc/m) | 50 |
| pulse delay (sec) | 2 | sartp (PSI) | 1 |
| maxpre (PSI) | 500 | sartf (cc/m) | 500 |
| pulse width (sec) | 0.2 | | |

### Light Transmission Measurements

A radiometer (Model IC 1700, International Light, Newburyport, Mass.) was used to measure the amount of light transmission through the samples of ePTFE membrane. A black tube, approximately the same diameter as the outer diameter of the light receiving sensor, Model SED 033, was clamped onto the light sensor and protruded approximately 13.3 cm from the receiving face of the sensor. The light source, a Sylvania Reflector 50W, 120V bulb, was mounted directly across from the light sensor approximately 28.6 cm from the receiving face of the light sensor. The radiometer was calibrated by placing a cap over the end of the tube protruding from the light sensor to set the zero point, and removing the cap and turning on the light source with nothing between the light source and the light sensor to set the 100% point. After calibrating the radiometer, the sterilization wrap material was held approximately 25.4 cm in front of the light sensor. The percent transmission of light displayed on the IC 1700 radiometer was recorded. The average of the three measurements was used.

### Standard Test Method for Stiffness of Fabric Using Blade/Slot Procedure

Materials were tested generally following the guidelines of ASTM D6828-02, Standard Test Method for Stiffness of Fabric Using Blade/Slot Procedure. Conditioning protocols identified as paragraph 9.1 were not applicable/or followed for these tests. A Handle-O-Meter model 211-3000, Thwing-Albert Instrument Company, Philadelphia, Pa., was used for stiffness testing. Samples of each material were cut into 100 mm×100 mm squares and tested in the machine and cross machine directions both face up and face down for a total of four tests per square sample. This was repeated 5 times for each material type for a total of 20 test measurements which were averaged and reported as material stiffness in grams.

The following non-limiting examples are provided to further illustrate the present invention.

### EXAMPLES

### Example 1

An ePTFE membrane having an mass area of approximately 3.0 g/m², a gurley of approximately 10 seconds, a bubble point of approximately 690 kPa, a thickness of approximately 8 um and a density of approximately 0.4 g/cc, was laminated to bi-component backer having a mass area of approximately 17 g/m² using a hot roll lamination process as described in FIG. 3. The bi-component backer was a PE sheath over a PET core, (S111) available from HDK Industries Inc., Rogersville, Tenn. The ePTFE membrane was laminated as shown in FIG. 3, with the hot roll 10 set to a temperature of 170° C. and the speed set to 60 m/minute. The elastomeric roll 20 was silicone rubber. The resulting laminate was then characterized by measuring various properties in accordance with tests described herein. Laminate properties for the sterilization packaging material made in this example appear in Table 3.

An approximately 1 m×1 m sheet of the laminate describe in Example 1 was loosely folded using envelope wrap technique, and placed in a steam sterilizer, Kuhlman Pharmapro KG2222 Sterilizer, Prometco Inc, Woodinville, Wash., at 121° C. for 30 minutes. The tensile break load properties for this laminate before and after sterilization appear in Table 4.

### Example 2

An ePTFE membrane having an mass area of approximately 8 g/m², a gurley of approximately 6.4 seconds, a bubble point of approximately 200 kPa, a thickness of approximately 20 um and a density of approximately 0.5 g/cc, was laminated to a bi-component backer having a mass area of approximately 17 g/m² using a hot roll lamination process as described in FIG. 3. The bi-component backer was a PE sheath over a PET core, (S111) available from HDK Industries Inc., Rogersville, Tenn. The ePTFE membrane was laminated as shown in FIG. 3, with the hot roll 10 set to a temperature of 170° C. and the speed set to 60 m/minute. The elastomeric roll 20 was silicone rubber. This laminate was then characterized by measuring various properties in accordance with the tests described herein. Laminate properties for the sterilization packaging material made in this example appear in Table 3.

The ePTFE membrane described in this example was evaluated using the particle shed analysis technique described herein. The average rate of particle measured greater than 0.5 um was 148. This is an average of two tests and demonstrates that the ePTFE membrane in non-linting.

An approximately 1 m×1 m sheet of the laminate described in Example 2 was folded loosely and placed into a vapor hydrogen peroxide sterilizer, STERIS MD880, Steris Corp., Mentor, Ohio, at 30° C. for 12 sterilization pulses with pulse hold totals of 5 minutes (60 minutes total). The tensile break load properties for this laminate before and after sterilization appear in Table 4.

### Example 3

An ePTFE membrane having an mass area of approximately 8 g/m², a gurley of approximately 6.4 seconds, a bubble point of approximately 200 kPa, a thickness of approximately 20 um and a density of approximately 0.5 g/cc, was laminated to bi-component backer having a mass area of approximately 42 g/m² oz/yd² using a hot roll lamination process as described in FIG. 3. The bi-component backer was a PE sheath over a PET core, (S111) available from HDK Industries Inc., Rogersville, Tenn. The ePTFE membrane was laminated as shown in FIG. 3, with the hot roll 10 set to a temperature of 170° C. and the speed set to 60 m/minute. The elastomeric roll 20 was silicone rubber. This laminate was then characterized by measuring various properties in accordance with the tests described herein. Laminate properties for the sterilization packaging material made in this example appear in Table 3.

TABLE 3

| | Example 1 | Example 2 | Example 3 | Example 4 | Example 5 | Example 6 |
|---|---|---|---|---|---|---|
| Gurley Number (sec) | 12.6 | 17.5 | 13.1 | 18.9 | | 17.5 |
| Bubble point (KPa) | 738 | 228 | 217 | 241 | 738 | 228 |
| LRV | 6.1 | | 6.0 | 6.0 | | |
| % light transmission | | | | | 60 | 34 |

The values reported in Table 3 show that the sterilization packaging material made according to the examples has sufficient permeation to allow the gas sterilant through the material, a high bubble point pressure or tight pore size, is bacteria impermeable having a LRV greater than 5.5 as define herein, and that is has sufficient light transmission to allow for the identification of items including indicator color change and reference identification codes through the material.

An approximately 1 m by 1 m sheet of the laminate described in Example 3 was wrapped around a sterilization tray using the envelope technique as described in AAMI ST46. The wrapped tray was placed into the TSO₃ Model 125L Ozone Sterilizer, available from TSO₃ Inc, Quebec, Canada, and a standard sterilization cycle was run. The chamber was pumped down to approximately 1 Torr and then water vapor was admitted into the chamber until a pressure of approximately 31 to 44 Torr was achieved at which point Ozone at a concentration of approximately 160 to 200 mg/L (NTP) was injected into the chamber until a dose of ozone of 85 mg/L was achieved, corresponding to a pressure of 400 to 500 Torr. The chamber was maintained at this condition for approximately 15 minutes. The chamber was then pumped back down to 1 Torr and the cycle was repeated. The sterilized laminate and a retain of the laminate not run through the ozone sterilization cycle were then evaluated for LRV and both had an LRV of 6.0. There was no reduction in LRV and the ozone sterilized laminate still stopped all the challenge spores as shown. The tensile break load properties for this laminate before and after sterilization appear in Table 4.

TABLE 4

| | Example 1 | Example 2 | Example 3 | Example 4 |
|---|---|---|---|---|
| Longitudinal | | | | |
| Max Load (N) | 38.6 | 28.0 | 79.4 | 143.7 |
| % Elongation at Max | 34.7 | 27.8 | 40.2 | 41.7 |
| Load | | | | |
| Transverse | | | | |
| Max Load (N) | 20.7 | 18.2 | 43.3 | 60.9 |
| % Elongation at Max | 45.3 | 55.6 | 17.648 | 77 |
| Load | | | | |

After Sterilization

| | | | |
|---|---|---|---|
| Longitudinal | | | |
| Max Load (N) | 43.4 | 31.3 | 37.2 |
| % Elongation at Max | 4.8 | 30.4 | 67.1 |
| Load | | | |
| Transverse | | | |
| Max Load (N) | 25.3 | 20.1 | 19.7 |
| % Elongation at Max | 47.6 | 55.6 | 36.5 |
| Load | | | |

The values reported in Table 4 demonstrate that the sterilization packaging materials made in accordance with the examples of the present invention have sufficient strength to be used in a variety of gas sterilization systems and that the strength after sterilization is sufficient for these types of applications. The maximum load values in Example 1 were greater for the post sterilized material because of some shrinkage that occurred during the steam sterilization cycle.

The material made in accordance with Example 3 and Example 4, Tyvek 1073B, DuPont de Nemours, Wilmington, Del., KC 600 One Step, as well as KC100 One Step, Kimberly Clark, Dallas, Tex., were all tested for stiffness generally following the method described in ASTM D6828-02, Standard Test Method for Stiffness of Fabric Using Blade/Slot Procedure. A 0.25″ slot width and a 1,000 g load was used for all of the testing. Result of this testing appear in Table 5.

TABLE 5

| Sample | Material Stiffness | LRV Value |
|---|---|---|
| Tyvek 1073B | 1650 | 5.2* |
| KC600 | 1048 | 2** |
| KC100 | 119 | 2** |
| Example 3 | 221 | 6.0 |
| Example 4 | 570 | 6.0 |

*This LRV Value is published data by DuPont de Nemours Inc. and was located at www.dupont.com/Medical Packaging/en US/assets/downloads
**These values were obtained from a Kimberly Clark Technical Data Sheet.

The values reported in Table 5 demonstrate that the sterilization packaging materials made in accordance with Example 3 and 4 of the present invention have less stiffness than commercially available sterilization packaging material but are stiff enough to be used as a sterilization wrap material. The unit measurements for material stiffness values are reported in grams.

### Example 4

An ePTFE membrane having an mass area of approximately 6 g/m², a gurley of approximately 13 seconds, a bubble point of approximately 324 kPa, a thickness of approximately 10 um and a density of approximately 0.7 g/cc, was laminated to bi-component backer having a mass area of approximately 68 g/m² using a hot roll lamination process as described in FIG. 3. The bi-component backer was a PE sheath over a PET core, (S111) available from HDK Industries Inc., Rogersville, Tenn. The ePTFE membrane was laminated as shown in FIG. 3, with the hot roll 10 set to a temperature of 170° C. and the speed set to 60 m/min. The elastomeric roll 20 was silicone rubber. This laminate was then characterized by measuring various properties in the manner described above. Laminate properties for the sample made in this example appear in Table 3, and the tensile break load properties for this laminate appear in Table 4.

The material made in accordance with Example 3 and Example 4, Tyvek 1073B, DuPont de Nemours, Wilmington, Del., KC 600 One Step, as well as KC100 One Step, Kimberly Clark, Dallas, Tex., were all tested for stiffness generally following the method described in ASTM D6828-02, Standard Test Method for Stiffness of Fabric Using Blade/Slot Procedure. A 0.25″ slot width and a 1,000 g load was used for all of the testing. Result of this testing appear in Table 5.

### Example 5

An ePTFE membrane having an mass area of approximately 1 g/m², a gurley of approximately 2.6 seconds, a bubble point of approximately 276 kPa, a thickness of approximately 2.5 um and a density of approximately 0.4 g/cc, was laminated to a plastic netting material using a Carver Press, Model C from Carver, Inc., Wabash, Ind. The netting material was a polyester apertured film KX225NAT-S available from Delstar Inc., Middletown, Del. The ePTFE membrane was laminated as shown in FIG. 4, with the top hot plate 50 set to a temperature of 160° C. The ePTFE membrane 2 was placed over the netting 13 and a layer of 2 mil polyimide film 8 was placed over the ePTFE membrane as shown in FIG. 9. A silicone rubber sheet 9 was placed on the bottom plate. The bottom plate of the Carver Press was raised and pressed against the top plate to a pressure of approximately 2.2 kN and held for approximately 2 seconds before the bottom plate 60 was released. This laminate was then characterized by measuring various properties in the manner described above. Laminate properties for the sample made in this example appear in Table 3.

### Example 6

The laminate as described in Example 2 was coated with Silicone rubber, RTV 615 from GE, Fairfield, Conn. The RTV 615 was cast onto a 50 um thick biaxially oriented polypropylene plastic film to a thickness of approximately 75 um. A square approximately 7.6 cm×7.6 cm was cut out from the coated plastic film and placed onto the support layer side of the laminate and allowed to cure overnight. The plastic film was peeled away from the silicone and the laminate was placed in an oven at 100° C. for one hour to ensure a complete cure. The area of the laminate where the cast RTV 615 was applied was translucent.

### Example 7

To demonstrate the translucent properties and more specifically that text and color can be visually discerned through the sterilization packaging material of the present invention, the word Size “#”, in bolded Arial text format was printed onto a standard A4 sheet of copy paper in increasing font size with the “#” corresponding to the font size. Font sizes from 10 to 28 in increments of 2 as well as font size 36 and 48 were printed on a single text test sheet. In addition, color circles approximately 30 mm and 18 mm in diameter in red, yellow, black and blue were printed for the color test sheet. The printed text and color test sheets were placed in a stainless steel tray that was 42 cm long by 25 cm wide by 6.5 cm deep, 18-8-SS-NSF, T304 Polar Ware Company, Sheboygan, Wis. The sterilization packaging material made in accordance with Example 1 was wrapped over the open top of the tray and the text and color tests sheets were observed through the sterilization packaging material. The print in all font sizes could be visually read through the sterilization packaging material and all four colors circles could be observed and discerned. The same test was performed by placing the materials described in Examples 5 and 6 over the open top of the tray, and all of the text font sizes could be read, and the colors could be discerned through the material. The material described in Example 5 was layered and placed over the open top of the tray, and all the text font sizes could be read, and the colors could be discerned through two layers of this material.

### Example 8

The ePTFE membrane described in Example 2 as well as the support material S305 described in Example 4 were both evaluated for particle shed analysis using the Helmke Tumble Test method described herein. The ePTFE membrane had an average particle count of 148 and the S305 support material had an average particle count of 10144. This test demonstrates that the ePTFE membrane provides a non-linting surface for the sterilization packaging material.

What is claimed is:

1. A sterilization packaging material for gas sterilization systems comprising:
(a) a first bacteria impermeable, gas permeable, non-linting microporous membrane layer; and
(b) a support layer attached to said first microporous membrane layer, said support layer providing physical support to the microporous membrane layer and increasing the stiffness of the sterilization packaging material while being compatible with gas sterilization systems.

2. The sterilization packaging material of claim 1 further comprising a second bacteria impermeable, gas permeable, non-linting microporous membrane layer attached to a side of said support layer opposite to said attached first microporous membrane layer.

3. The sterilization packaging material of claim 1 wherein said microporous membrane layer comprises expanded polytetrafluoroethylene (ePTFE).

4. The sterilization packaging material of claim 1 wherein said support layer comprises ePTFE membranes.

5. The sterilization packaging material of claim 4 wherein said support layer comprises a polyethylene.

6. The sterilization packaging material of claim 5 wherein said support layer is a non-woven comprising a bi-component fiber with a polyethylene sheath over a polyester core.

7. The sterilization packaging material of claim 1 wherein said support layer is an ePTFE membrane or an FEP netting material.

8. The sterilization packaging material of claim 1 wherein said microporous membrane layer and said support layer are translucent.

9. The sterilization packaging material of claim 1 wherein a portion of said microporous membrane layer and said support layer is made translucent by partially imbibing a polymer into pores of the microporous membrane layer.

10. The sterilization packaging material of claim 1 wherein a portion of said microporous membrane layer and said support layer are made translucent by compression of said microporous membrane layer to said support layer.

11. The material of claim 1 wherein the gas sterilization system is an ozone sterilization system.

12. The material of claim 1 wherein the gas sterilization system is ethylene oxide sterilization system

13. A method of article sterilization comprising the steps of:
(a) placing an article to be sterilized in a sterilization enclosure;
(b) enclosing said sterilization enclosure with at least one layer of the sterilization packaging material of claim 1;
(c) placing said enclosed sterilization enclosure in a gas sterilization system; and
(d) running the sterilization cycle at least once.

14. The method of claim 13 wherein the gas sterilization system is an ozone sterilization system.

15. The method claim 1 wherein the sterilization packaging material has a translucent portion.

16. The method of claim 15 further comprising the step 1(a) placing an effective sterilization indicator in said sterilization enclosure which can be visualized throughout the translucent portion of the sterilization packaging material following the sterilization cycle.

17. A sterilization package comprising:
(a) an enclosure for holding items to be sterilized; and
(b) the sterilization packaging material of claim 1 which seals or wraps around the enclosure.

18. The sterilization package of claim 17 wherein the enclosure is a tray and the sterilization packaging material wraps around the tray.

19. The sterilization package of claim 17 wherein the enclosure is a container and the sterilization packaging material seals an opening of the container.

20. The sterilization package of claim 17 wherein the enclosure is a pouch of the sterilization packaging material.
